(19) United States
(12) Patent Application Publication
(10) Pub. No.: US 2006/0200245 A1
(43) Pub. Date: Sep. 7, 2006

(54) Materials, devices, and methods for in-situ formation of composite intervertebral implants

(75) Inventor: Hai Trieu, Cordova, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI HOLDINGS, INC.

(21) Appl. No.: 11/072,972

(22) Filed: Mar. 7, 2005

(51) Int. Cl. A61F 2/44; A61F 2/02

(52) U.S. Cl. 623/17.16; 623/23.74

(57) ABSTRACT

An intervertebral disc repair devices is disclosed that includes a porous matrix and a polymerizable material. The intervertebral disc repair device is advantageous because it may be injected through a small annulus defect, it can form an implant larger than the annulus defect for improved expulsion resistance, it has increased toughness and durability because of the porous matrix, and it conforms to the partially or fully evacuated disc space during insertion or packing.

### FIELD OF THE INVENTION

The present invention relates generally to intervertebral disc reconstruction or repair devices and methods and more specifically to intervertebral disc reconstruction or repair devices and methods comprising a porous matrix and polymerizable material.

### BACKGROUND OF THE INVENTION

The intervertebral disc functions to stabilize the spine and to distribute forces between vertebral bodies. The intervertebral disc is composed of three structures: the nucleus pulposus, the annulus fibrosis, and two vertebral end plates. These components work to absorb the shock, stress, and motion imparted to the human vertebrae. The nucleus pulposus is an amorphous hydrogel with the capacity to bind water. The nucleus pulposus is maintained within the center of an intervertebral disc by the annulus fibrosis, which is composed of highly structured collagen fibers. The vertebral end plates, composed of hyalin cartilage, separate the disc from adjacent vertebral bodies and act as a transition zone between the hard vertebral bodies and the soft disc.

Intervertebral discs may be displaced or damaged due to trauma or disease. Disruption of the annulus fibrosis may allow the nucleus pulposus to protrude into the vertebral canal, a condition commonly referred to as a herniated or ruptured disc. The extruded nucleus pulposus may press on a spinal nerve, which may result in nerve damage, pain, numbness, muscle weakness, and paralysis. Intervertebral discs may also deteriorate due to the normal aging process. As a disc dehydrates and hardens, the disc space height will be reduced, leading to instability of the spine, decreased mobility and pain.

One way to relieve the symptoms of these conditions is by surgical removal of a portion or the entire intervertebral disc. The removal of the damaged or unhealthy disc may allow the disc space to collapse, which would lead to instability of the spine, abnormal joint mechanics, nerve damage, as well as severe pain. Therefore, after removal of the disc, adjacent vertebrae are typically fused to preserve the disc space. Spinal fusion involves inflexibly connecting adjacent vertebrae through the use of bone grafts or metals rods. Because the fused adjacent vertebrae are prevented from moving relative to one another, the vertebrae no longer rub against each other in the area of the damaged intervertebral disc and the likelihood of continued irritation is reduced. Spinal fusion, however, is disadvantageous because it restricts the patient's mobility by reducing the spine's flexibility, and it is a relatively invasive procedure.

Attempts to overcome these problems have led researchers to investigate the efficacy of implanting an artificial intervertebral disc to replace, completely or partially, the patient's damaged intervertebral disc. Disc replacement surgery generally involves removing the disc or damaged portion thereof and placement of an artificial disc in the evacuated disc space. Some desirable attributes of a hypothetical implantable disc include axial compressibility for shock absorbance, excellent durability to avoid future replacement, minimally invasive placement of the artificial disc to reduce post-operative discomfort, and biocompatibility. Existing artificial intervertebral discs include, for example, mechanically based (e.g. comprising rotational surfaces or springs), polymer based, and biopolymer based artificial discs.

Among the polymer based artificial intervertebral discs are several devices that utilize a flowable polymer. One example of such a device is U.S. Pat. No. 3,875,595, incorporated herein by reference in its entirety, which discloses an intervertebral disc prosthesis comprising a flexible bladder-like member that is inserted into the evacuated disc space. The prosthesis is anchored to the two adjacent vertebrae through the use of studs inserted into the bone and filled with a fluid, plastic, or hydrogel until the bladder expands to fill the evacuated disc space.

In another example, U.S. Pat. No. 6,264,659, incorporated herein by reference in its entirety, the nucleus pulposus is removed. A thermoplastic material is heated until its viscosity is sufficiently reduced to allow it to be injected under pressure into the annulus fibrosis. The thermoplastic then cools to body temperature and stiffens but retains sufficient resiliency to provide cushioning of the vertebrae and joint movement.

U.S. Pat. No. 6,187,048, incorporated herein by reference in its entirety, discloses an intervertebral disc implant wherein the nucleus pulposus is removed and a flowable polymer is injected into the evacuated annulus fibrosis. The flowable polymer is caused to cure in situ, forming a shaped, resiliently deformable prosthesis.

U.S. Pat. No. 6,140,452, incorporated herein by reference in its entirety, discloses an intervertebral disc implant wherein a multi-part polyurethane biocompatible polymer is injected into the evacuated disc space, preferably through the use of a cannula and arthroscope. The flowable composition then is cured in place.

The description herein of problems and disadvantages of known apparatus, methods, and devices is not intended to limit the invention to the exclusion of these known entities. Indeed, embodiments of the invention may include one or more of the known apparatus, methods, and devices without suffering from the disadvantages and problems noted herein.

### SUMMARY OF THE INVENTION

An improved artificial intervertebral disc repair device would be advantageous. A number of advantages associated with the present invention are readily evident to those skilled in the art, including economy of design and resources, ease of use, cost savings, etc.

A feature of an embodiment of the invention includes an intervertebral disc repair device comprising a porous matrix and a polymerizable material. The porous matrix preferably includes but is not limited to mesh, sheeting, tubing, fabric, sponges, or any other appropriate biocompatible porous material. The porous matrix may be synthetic, natural, or a combination thereof. The polymerizable material may be any biocompatible polymer with the ability to cure in situ. Preferred polymerizable materials include, but are not limited to, two-part polymers and water, heat, and light activated polymers.

The polymerizable material may be applied to the porous matrix before or after insertion of the porous matrix into the evacuated intervertebral disc space. The polymerization reaction may be initiated by body fluids, saline solution, sterile water, light, body heat, external heat, injection of the complementary part of a two-part polymer, or by any other suitable initiation method. The polymerizable material preferably is allowed to cure in situ.

In accordance with another feature of an embodiment of the invention, there is provided a method of making an intervertebral disc repair device that includes contacting a porous matrix with a polymerizable material, and causing the polymerizable material to polymerize. In preferred embodiments, the porous matrix is contacted with the polymerizable material, or a portion thereof, prior to insertion into the evacuated intervertebral disc space. In other preferred embodiments, the porous matrix is contacted with the polymerizable material, or a portion thereof, after insertion into the evacuated intervertebral disc space.

In yet another feature of an embodiment of the invention, there is provided a method of implanting an intervertebral disc repair device that includes providing a porous matrix, optionally contacting the porous matrix with a polymerizable material, and compressing the porous matrix to reduce at least one of its three dimensional dimensions. The method then includes forming a passageway to an intervertebral disc space that is either fully or partially evacuated, and inserting the compressed porous matrix into the intervertebral disc space. The method can be completed by causing the polymerizable material to polymerize in situ to form an intervertebral disc repair device.

Still further features and advantages of the present invention are identified in the ensuing description, with reference to the drawings identified below.

### BRIEF DESCRIPTION OF THE DRAWINGS

The purpose and advantages of the present invention will be apparent to those of ordinary skill in the art from the following detailed description in conjunction with the appended drawings in which like reference characters are used to indicate like elements, and in which:

FIG. 1 is a cross sectional drawing of the intervertebral disc.

FIG. 2 is an illustration of the intervertebral disc and its placement in the spine.

FIG. 3 is an illustration of the porous matrix.

FIG. 4 is an illustration of a method of inserting the porous matrix into the evacuated disc space.

FIG. 5 is an illustration of a method of inserting the porous matrix and injecting the polymerizable material into the evacuated disc space.

### DETAILED DESCRIPTION OF THE INVENTION

The following description is intended to convey a thorough understanding of the present invention by providing a number of specific embodiments and details involving use of a porous matrix and polymerizable material for intervertebral disc reconstruction or repair. It is understood, however, that the present invention is not limited to these specific embodiments and details, which are exemplary only. It is further understood that one possessing ordinary skill in the art, in light of known systems and methods, would appreciate the use of the invention for its intended purposes and benefits in any number of alternative embodiments, depending upon the specific design and other needs.

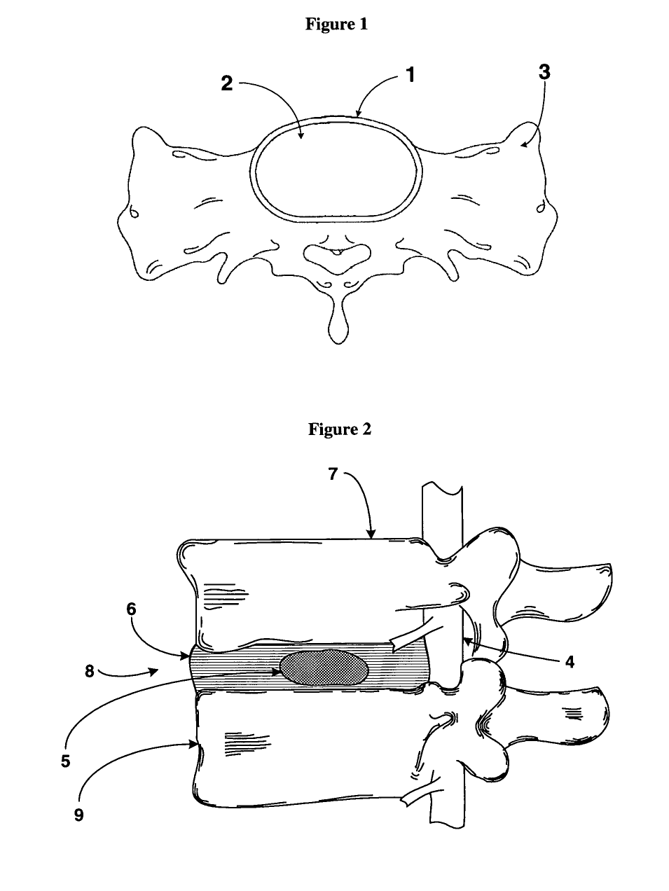

Referring now to FIG. 1, the intervertebral disc contains the annulus fibrosis 1, which surrounds the nucleus pulposus 2 and contacts vertebrae 3. FIG. 2 further illustrates the location of the annulus fibrosis 6 around the nucleus pulposus 5. Vertebrae 7 and 9 are adjacent to intervertebral disc 8. Annulus fibrosis 6 also contacts spinal cord 4.

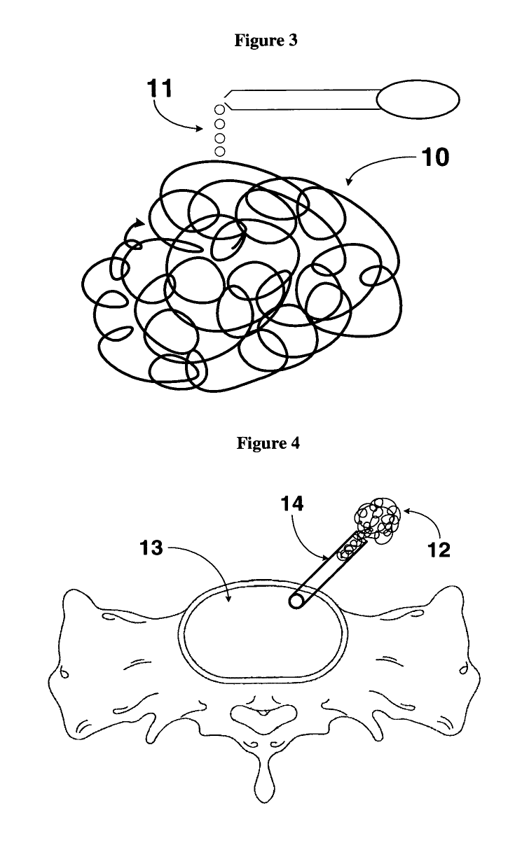

In an embodiment of the present invention, as illustrated in FIGS. 3 and 4, the porous matrix 10 initially is saturated with the polymerizable material 11. The porous matrix including the polymerizable material 12 then is inserted into the partially or completely evacuated disc space 13 by means of an exemplary instrument 14. Finally, the porous matrix including the polymerizable material 12 is allowed to cure in place in the evacuated disc space 13. In one preferred embodiment of the present invention, the polymerization reaction is initiated by the subsequent injection of, for example, saline solution, water, the complementary part of a two-part polymer, application of light, application of heat, or any other initiation process. In another preferred embodiment of the present invention, body fluids or body heat initiate the polymerization reaction.

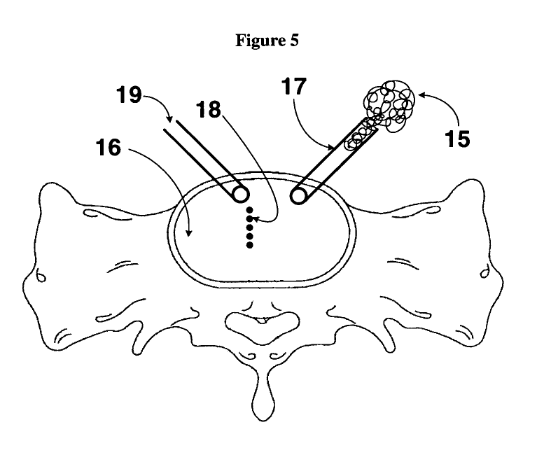

In another embodiment of the present invention, as illustrated in FIG. 5, the porous matrix 15 may be inserted into the partially or completely evacuated disc space 16 by means of an exemplary instrument 17. The porous matrix preferably is inserted without pre-contacting with the polymerizable material. Only after insertion of the porous matrix 15 is the polymerizable material 18 inserted by means of an exemplary instrument 19. In a preferred embodiment of the present invention, the polymerization reaction is initiated by the subsequent injection of, for example, saline solution, water, the complementary part of a two-part polymer, application of light, application of heat, or any other initiation process. In another preferred embodiment of the present invention, body fluids or body heat initiate the polymerization reaction. In yet another preferred embodiment, the porous matrix 15 is contacted with water or saline solution and then inserted into the evacuated disc space 16, followed by injection of the polymerizable material 18.

Any porous matrix may be used in the invention so long as it is capable of supporting the polymerizable material and forming a suitable intervertebral disc repair device together with the polymerized material. Porous matrix include, but are not limited to, mesh, sheeting, tubing, fabric, sponges, woven fabrics, non-woven mesh, braided tubing, three-dimensional woven structures, or any other appropriate bio-compatible porous material. The porous matrix may be synthetic, natural, or a combination thereof. Suitable materials for the porous matrix include woven, braided, and non-woven materials, which may be fibrous or non-fibrous. For fibrous materials, the size of the fibers and the fiber density can be varied as appropriate to control mechanical strength. For non-fibrous materials (e.g. plastics films), perforations of an appropriate size may be provided. Suitable materials for forming the porous matrix include, but are not limited to, polyethylenes (which may be ultra high molecular weight polyethylenes), polyesters, polyurethanes, polyesterurethane, polyester/polyol block copolymers, poly ethylene terephthalate, polytetrafluoro ethylene polyesters, nylons, polysulphanes, cellulose materials, polyaramids, carbon or glass fibers, polyvinyl chlorides, stryrenic resins, polypropylenes, polycarbonates, acrylonitrile-butadiene-styrene (“ABS”), acrylics, styrene acrylonitriles, and mixtures, copolymers, and combinations thereof. See, for example, “Guide to Medical Plastics”, pages 41-78 in Medical Device & Diagnostic Industry, April, 1994.

Any polymerizable material may be used in the invention so long as it is capable of forming a suitable intervertebral disc repair device upon polymerization. The polymerizable material may be used in any applicable state, for example, as a liquid, gel, paste, suspension, powder, or granules. The polymerizable material may be a monomer, oligomer, or material capable of undergoing cross-linking either by itself, or with the aid of cross-linking agents or external force (e.g., heat, light, etc.). One who is skilled in the art will recognize that the state in which the polymerizable material is used for purposes of this invention may be chosen to correspond with the particular conditions expected during disc reconstruction or repair. For example, where it is feared that the polymerizable material may flow out of the disc space where it is intended to be implanted, it may be advantageous to apply the polymerizable material in a non-flowing, or solid, state. In other situations, for example where the polymerizable material is to be injected into the disc space, it may be desirable to apply the polymerizable material in a liquid state.

In accordance with one embodiment of the present invention, the polymerizable material is a water-activated polymer. In one preferred embodiment, contact with body fluids after implantation initiates the polymerization reaction. In another preferred embodiment, water or saline solution may be injected into the porous matrix after implantation. In yet another preferred embodiment, the porous matrix can be soaked in water or saline solution, implanted into the partially or fully evacuated disc space, and then injected with the water-activated polymer. In yet another preferred embodiment, the porous matrix can be contacted with water or saline solution, the water-activated polymerizable material, and then implanted into the partially or fully evacuated disc space before the polymerizable material fully cures.

In one preferred embodiment, the water activated polymerizable material may be a polyfunctional isocyanate based prepolymer wherein water can be used to effect polymerization by causing the formation of urea linkages. Blocked isocyanate prepolymers that, on crosslinking with an active prepolymer, can polymerize about or below body temperature also may be used. An example of this type of system is a polyurethane resin containing blocked isocyanate groups based on toluene diisocyanate and p-isononyl phenol reacted with a polyfunctional amine terminated polymer such as polyalkylene oxide amine terminated polymer (e.g. JEFFAMINE D2000®, commercially available from Texaco Chemicals, San Francisco, Calif.). The hydrophilicity of these systems may be varied by reaction of the blocked isocyanate resin with polyfunctional amine terminated polymers that contain a high proportion of ethylene oxide (e.g. JEFFAMINE ED-600®, commercially available from Texaco Chemicals, San Francisco, Calif.). Alternatively the blocked isocyanate polyurethane prepolymers may be prepared using polyols with high ethylene oxide content.

Another alternative is to use siloxanes comprising functional groups that allow polymerization of the siloxanes with water to occur (e.g. alkoxy, acyloxy, amido, oximo or amino groups). Acyloxy, acetoxy and alkoxy functionalities are most frequently employed. The number of siloxane groups may be determined such that the cured polymer is a resiliently deformable material.

In another embodiment of the present invention, the polymerizable material may be a two-part polymerizable material. In a preferred embodiment, the two-part polymerizable material forms a polyurethane and has as Part I an isocyanate-functional polyurethane pre-polymer (optionally referred to as an “quasi-polymer”). The quasi-polymer of Part I typically includes a polyol component in combination with a hydrophobic additive component and an excess of an isocyanate component. Part II of the two-part polymerizable material may include long-chain polyols, chain extenders, or cross-linkers, together with other ingredients (e.g., catalysts, stabilizers, plasticizers, antioxidants, dyes and the like). Such adjuvants or ingredients may be added to or combined with any other component thereof either prior to or at the time of mixing, delivery, and/or curing.

The isocyanate component may be provided in any suitable form, examples of which include 2,4′-diphenylmethane diisocyanate, 4,4′-diphenylmethane diisocyanate, toluene diisocyanates, and mixtures or combinations of these isomers, optionally together with small quantities of 2,2′-diphenylmethane diisocyanate. Other examples include aromatic polyisocyanates and their mixtures or combinations, such as are derived from phosgenation of the condensation product of aniline and formaldehyde. An isocyanate that has low volatility, such as diphenylmethane diisocyanate, rather than more volatile materials such as toluene diisocyanate, may be used. An example of a particularly suitable isocyanate component is the 4,4′-diphenylmethane diisocyanate (“MDI”), preferably provided in liquid form as a combination of 2,2′-, 2,4′- and 4,4′-isomers of MDI.

The polyol component may be provided in any suitable form as well. As used herein, the term “polyol” includes virtually any functional compound having active hydrogens in accordance with the well-known Zerevitinov test, as described for instance in Chemistry of Organic Compounds by Carl R. Noller, Chapter 6, pp. 121-122 (157). Thus, for example, amine terminated polyethers and polyolefins, thiols, polyimines, and polyamines also can be used as polyols in the present invention. Suitable polyols for use in preparing a composition of this invention also include polyalkylene ethers derived from the condensation of alkylene oxides (e.g., ethylene oxide, propylene oxide, and blends thereof), as well as tetrahydrofuran based polytetramethylene ether glycols, polycaprolactone polyols, polycarbonate polyols and polyester polyols. Examples of suitable polyols include polytetrahydrofuran polyol (“PTHF”, also known as polytetramethylene oxide (“PTMO”) or polytetramethylene ether glycol (“PTMEG”)).

In a further preferred embodiment of the present invention, the two-part polymerizable material forming a polyurethane contains one or more, and more preferably two or more, biocompatible catalysts that can assist in controlling the curing process during one or more of the following periods: (1) the induction period, (2) the setting period, and finally, (3) the final cure of the biomaterial. Together these three periods, including their absolute and relative lengths, and the rate of acceleration or cure within each period, determine the cure kinetics or profile. Examples of suitable catalysts include tin compounds (such as tin esters, tin alkylesters, and tin mercaptides), amines, such as tertiary amines and the like. An example of a suitable catalyst system is a combination of a tin catalyst (e.g., COTIN 222®, available commercially from Cascam Company, Bayonne, N.J.) and a tertiary amine (e.g., DABCO(TEDA)®, a triethylene diamine catalyst available commercially from Air Products, Allentown, Pa.). These components can be used in any suitable ratio, e.g., between about 1:1 parts and about 1:5 parts of the tin catalyst and the diamine, respectively.

In yet another further preferred embodiment of the present invention, the two-part polymerizable material forming a polyurethane comprises a diisocyanate, a polyalkylene oxide, and low molecular diols as chain extenders. The final polymer having a hard segment content of about 25 to about 50% by weight, and preferably of about 30 to about 40% by weight, based on the weight of the diisocyanate and chain extender. Optionally, one or more catalysts may be incorporated into one or more components of the biomaterial in order to polymerize the biomaterial in the physiological environment within a desired length of time. Preferably, biomaterials of the present invention are able to polymerize (i.e., to the point where distraction means can be removed and/or other biomaterial added), within 5 minutes or less, and more preferably within on the order of 3 minutes or less.

In another preferred embodiment of the present invention, the two-part polymerizable material may comprise mixtures of poly(hydroxyalkyl(meth)acrylates) and poly(alkyl(meth)acrylates) crosslinked using polyfunctional (meth)acrylate monomers or oligomers, (e.g. triethyleneglycol dimethacrylate). The reagent may be cured at low temperature by using a free radical initiator and an amine activator (e.g. benzoyl peroxide and dimethyl p-toluidene). Preferably the alkyl groups contain from 1 to 4 carbon atoms.

In another preferred embodiment of the present invention, the two-part polymerizable material may comprise a mixture of tetra and trifunctional epoxy resin blend reacted with multifunctional amines and amino terminated elastomers such as an epoxy terminated silane and an amino terminated nitrile rubber. The two-part polymerizable material may comprise a monomer oligomer or polymer that contains ethylenic unsaturation. The ethylenic unsaturation may be acrylic or methacrylic unsaturation.

In another embodiment of the present invention, polymer complexes may be used, e.g., complexes formed between the following polyanions, poly (sodium acrylate), poly (sodium vinyl sulphate) sodium poly phosphates, sodium polystyrene sulphonate and the following polycations: poly (N,N,N-trialkylammonioalkylacrylate), poly (N-alkylpyridinium) cation. There are several natural polymers that are capable of forming complexes. Anionic polymers include: sodium carboxymethyl cellulose, sodium cellulose sulphate, sodium alginate, and sodium hyaluronate. Cationic polymers include chitosan, quaternised chitosan, amino alkylated and subsequently quarternised cellulose, poly-L-lysine, and mixtures thereof.

Skilled artisans recognize other applicable polymerizable materials that may be utilized in accordance with the present invention. For example, polyurethanes, polyvinyl alcohols (PVA), PVA hydrogels, collagen, fibrin, heparin, keratin, albumin, silk, elastin, polyvinylpyrrolidone (PVP), PVP hydrogels, polyethylene glycol (PEG), PEG hydrogels, acrylamide hydrogels, acrylamide/maleic acid hydrogels, acrylic based hydrogels, polyalkylimines, silicone elastomers, polymethylmethacrylates, and mixtures and combinations thereof are all contemplated as suitable polymerizable materials. In general, any biologically inert polymerizable material may be used in the present invention.

In another embodiment of the present invention, the polymerizable materials are heat-activated to initiate polymerization. The temperature at which the polymerizable material is activated should be no more than about 20° C. above normal body temperature, and preferably is lower than or equal to body temperature, so that the internal heat of the body will cause the polymerization reaction to initiate. The heat-activated polymerizable material may either soak the porous matrix before insertion into the evacuated disc space or be injected into the porous matrix after the matrix has been inserted into the evacuated disc space.

In another embodiment of the present invention, the polymerizable materials are light activated to initiate polymerization. The light-activated polymerizable materials may be chosen such that the wavelengths of light used to initiate the polymerization reaction do not interact with or damage surrounding body tissues. For example, the polymerizable material may include any of the known photopolymerizable systems employed in photography (e.g., including ethylenically unsaturated compounds and photo-initiators), or those used in forming dental materials. A suitable material includes a one-part composition comprised of a polyfunctional urethane methacrylate and/or polyfunctional urethane acrylate and a polyfunctional acrylate resin. Urethane methacrylate is the product of the reaction of a diisocyanate with an OH-functional methacrylate, such as hydroxyethyl methacrylate for example. When a diisocyanate is used, the product is a urethane dimethacrylate; if an OH-functional acrylate is used, such as a hydroxyethyl acrylate, a difunctional acrylate is the result, similarly to the methacrylate. Such a urethane methacrylate or urethane acrylate, especially a urethane dimethacrylate is advantageous, because among other things it offers superior material properties such as great stiffness or low moisture absorption. Also possible is the use of a monomer prepared from the combination of triisocyanates or higher isocyanates with OH-functional acrylates or methacrylates, in which case these urethane methacrylates or urethane acrylates will have a functionality of 3 or more. Advantageously, the urethane methacrylate is a urethane dimethacrylate or urethane trimethacrylate and the urethane acrylate is a urethane diacrylate or a urethane triacrylate.

Other suitable photopolymerizable systems include those based on a multifunctional prepolymer mixture of 2,2-bis-(4-(2-hydroxy-3-methacryloyloxypropoxy)phenyl) propane, known commonly as “Bis-GMA.” These compositions typically include a photoinitiation system, and can include other fillers, diluents, additives, and the like. These systems are described in, for example, U.S. Pat. No. 4,102,856, U.S. Pat. No. 4,131,729, U.S. Pat. No. 3,730,947, and U.S. Pat. No. 6,339,113, the disclosures of each of which are incorporated herein by reference in their entirety.

Scavengers such as magnesium oxide may be advantageously employed if it is desired to reduce or eliminate any adverse effects of by-products of the polymerization reaction. Inhibitors also may be included to control the exothermic generation of heat in some systems such that the temperature of the implant material upon curing, does not increase much above that of body temperature. Suitable inhibitors may include p-methoxyphenol and hydroquinone.

Aspects of the invention also include methods of making an intervertebral disc repair device by contacting a porous matrix with a polymerizable material, and causing the polymerizable material to polymerize. In preferred embodiments, the porous matrix is contacted with the polymerizable material, or a portion thereof, prior to insertion into the evacuated intervertebral disc space. In other preferred embodiments, the porous matrix is contacted with the polymerizable material, or a portion thereof, after insertion into the evacuated intervertebral disc space.

When a two-part polymerization system is employed, as described above, one part of the polymerization system may be contacted with the porous matrix material prior to insertion into the fully or partially evacuated disc space. Upon insertion, the second part of the polymerization system may be contacted with the porous matrix and the first part of the polymerization system, thereby causing the material to polymerize and form an intervertebral disc repair device.

In one embodiment of the invention involving a two-part polymerization system, one of the parts may be a solid and the other part a liquid or slurry. The solid portion can be contacted with the porous matrix prior to insertion, and then the second component (liquid or slurry) injected after insertion. Alternatively, the porous matrix can be fabricated from a polymeric material that can serve as one of the components of a two-part polymerization system (including those where water is the second part). The polymeric material can be coated on an existing fabric or polymer (e.g., polymers used to make spun bonded or non-woven materials), or can be synthesized, and then spun or woven into a fabric-like material. The resulting porous matrix then can either be contacted prior to insertion or after insertion into the fully or partially evacuated disc space with the second component of the polymerization system to effect polymerization.

Other methods of forming the intervertebral disc repair device are described previously whereby the polymerization is effected by contact with water, light, heat, or other energy sources. Those skilled in the art will appreciate that the particular method used to form the intervertebral disc repair device is not particularly limited. Rather, the skilled artisan, using the guidelines provided herein, will recognize the various types of known and later discovered polymerization systems that can be used to achieve the advantages of the invention.

Other features of the invention include methods of implanting an intervertebral disc repair device. In one embodiment, the method includes providing a porous matrix, optionally contacting the porous matrix with a polymerizable material, and compressing the porous matrix to reduce at least one of its three dimensional dimensions. The method then includes forming a passageway to an intervertebral disc space that is either fully or partially evacuated, and inserting the compressed porous matrix into the intervertebral disc space. Any suitable instrumentation can be used to form the passageway, using techniques well known in the art. Particularly preferable instrumentation includes those capable of forming passageways using minimally invasive techniques, as will be appreciated by those skilled in the art. The porous matrix, optionally including the polymerizable material, then can be inserted into the partially or fully evacuated disc space using minimally invasive means, such as a relatively small cannula (e.g., 2-20 mm), and a flexible, semi-rigid push rod to push the matrix through the cannula.

The method can be completed by causing the polymerizable material to polymerize in situ to form an intervertebral disc repair device. If an additional liquid is to be added to effect polymerization, the liquid can be added using a suitable delivery instrument, such as a needle, or small cannula. If heat or light (or other energy source) is required to effect polymerization, micro-heaters, and/or endoscopic light sources can be inserted through the same delivery channel (e.g., cannula or other like device), or separately inserted delivery channel, to provide the requisite energy source.

The intervertebral disc repair device can be configured in practically any shape or size, and can be of suitable rigidity, by virtue of selecting the appropriate porous matrix material, to allow relatively easy insertion through the delivery channel. In addition, because the polymerization causes the porous matrix material to swell, the particular size and shape of the porous matrix material is not important, since the polymerized mass will fill the partially or fully evacuated disc space. Accordingly, the porous matrix material, either prior to or after contact with the polymerizable material, or portion thereof, can be an amorphous mass, a sphere, a cylinder, etc., or can be formed into such a shape prior to insertion into the passageway to the intervertebral disc space.

In another embodiment, there is provided a surgical kit. The surgical kit may contain the porous matrix and polymerizable material described herein. Preferably, the kit may contain several different porous matrices and polymerizable materials contained in appropriate containers so that a surgeon may conveniently select between the available porous matrices and polymerizable materials during surgery to repair or reconstruct an intervertebral disc. Additionally, the kit may contain other surgical instruments that may be advantageously utilized during surgery. For example, the kit may contain a trimming device. A trimming device may be used to form the porous matrix into the appropriate configuration and size to facilitate implantation into the disc space of the patient. Trimming devices include, for example, scissors, shears, knifes, and other cutting instruments.

The kit also may contain a device appropriate to inject the polymerizable material into the disc space of the patient, if that is how the polymerizable material is to be applied. For example, a suitable cannula, a double-barreled syringe or two single syringes with a connector for mixing may be included in the kit. One who is skilled in the art will appreciate other applicable injecting devices that may be included in the kit. The kit also may contain various general surgical tools useful to access the disc space or remove a portion or all of the intervertebral disc. A drill, drill tube, drill tube guide, reamer, guide pin, distractor, and distraction plug, for example, may be included in the surgical kit. Other generally useful surgical instruments that may be included in the kit include scalpels, cauterizing instruments, bandages, gauze, clamps, extraction tools, cannulas, medications, etc. One skilled in the art will appreciate the various tools that may be included in the surgical kit.

It will be readily apparent to those skilled in the art upon reading this description that the inventive intervertebral disc repair device provides advantages over liquid, semi-liquid, hydrogel systems, as well as fully solid disc repair systems. For example, there is little or no risk of leakage of the polymerizable material outside of the partially or fully evacuated disc space that can occur with liquid or semi-liquid systems. In addition, the porous matrix material provides much more flexibility than prior solid disc repair systems, thereby improving the ease of fabrication and insertion.

The invention now will be described in more detail by virtue of the following non-limiting examples.

### EXAMPLE 1

Polyethylene gauze served as the porous matrix. The polyethylene gauze was soaked with water and excess water was squeezed out. A curable NCO-terminated hydrophobic urethane pre-polymer composition containing toluene diisocyanate and an oxyethylene-based polyol as disclosed in U.S. Pat. No. 6,702,731, the disclosure of which is incorporated by reference herein in its entirety, then was applied to the wet gauze and polymerization allowed to proceed. The polyethylene gauze displayed advantageous properties including shape memory, elasticity, and other properties desirable for an intervertebral disc repair device.

### EXAMPLE 2

Woven polyester fabric serves as the porous matrix and is soaked with a water curable polymerizable material. The disk space is partially or fully evacuated by known surgical techniques, for example curettage, suction, laser nucleotomy, or chemonucleolysis. The soaked fabric is inserted into the partially evacuated disc space by use of a cannula and arthroscope and allowed to polymerize in the presence of body fluids.

### EXAMPLE 3

A woven polyethylene article is soaked with a photoactive polymer composition. The disc space is evacuated as in Example 1, and the soaked fabric is inserted as in Example 1. Then, a light source is inserted into the disc space for a period of time sufficient to activate the photoactive polymer to cause polymerization to proceed.

### EXAMPLE 4

A three-dimensional woven polyethylene article is prepared. The disc space is evacuated as in Example 1, and the three-dimensional article is inserted as in Example 1 to occupy a portion of the evacuated disc space. A polymethylmethacrylate bone cement composition (a powder and liquid combination) then is injected into the three-dimensional porous structure. The polymethylmethacrylate polymerizes to form a solid composite material in the disc space.

The invention has been described with reference to particularly preferred embodiments and examples. Those skilled in the art will appreciate that various modifications may be made to the invention without departing from the spirit and scope thereof.

What is claimed is:

1. An intervertebral disc repair device comprising:
a porous matrix;
and a polymerizable material.

2. The device as in claim 1, wherein the polymerizable material is injected into the porous matrix after the matrix is inserted into an evacuated disk space.

3. The device as in claim 1, wherein the porous matrix is contacted with the polymerizable material before the porous matrix is inserted into an evacuated disk space.

4. The device as in claim 1, wherein the polymerizable material is injected into the porous matrix after the matrix is inserted into an unevacuated disk space.

5. The device as in claim 1, wherein the porous matrix is contacted with the polymerizable material before the porous matrix is inserted into an unevacuated disk space.

6. The device as in claim 1, wherein the polymerizable material is selected from the group consisting of polyurethanes, polyvinyl alcohols (PVA), PVA hydrogels, collagen, fibrin, heparin, keratin, albumin, silk, elastin, polyvinylpyrrolidone (PVP), PVP hydrogels, polyethylene glycol (PEG), PEG hydrogels, acrylamide hydrogels, acrylamide/maleic acid hydrogels, acrylic based hydrogels, polyalkylimines, silicone elastomers, polymethylmethacrylates, and mixtures and combinations thereof

7. The device as in claim 1, wherein the polymerizable material is a water activated polymerizable material.

8. The device as in claim 7, wherein the water activated polymerizable material is a siloxane with a functional group that allows polymerization of the siloxane with water.

9. The device as in claim 8, wherein the water activated siloxane has alkoxy, acyloxy, acetoxy, amido, oximo, or amino functional groups.

10. The device as in claim 9, wherein the water activated polymerizable material is a polyfunctional isocyanate based polymerizable material.

11. The device as in claim 1, wherein the polymerizable material is a two-part polymerizable material.

12. The device as in claim 11, wherein the first part of the two-part polymerizable material is a solid.

13. The device as in claim 12, wherein the two-part polymerizable material forms a polyurethane and has as one part a diisocyanate or polymeric isocyanate and as the other part a polyol.

14. The device as in claim 13, wherein the two-part polymerizable material forms a silicone polyurethane.

15. The device as in claim 13, wherein the diisocyanate is selected from the group consisting of 2,4′-diphenylmethane diisocyanate, 4,4′-diphenylmethane diisocyanate, 2,2′-diphenylmethane diisocyanate, 2,4-toluene diisocyanate, 2,6-toluene diisocyanate, hexamethylene diisocyanate, dicyclohexylmethane diisocyanate, and mixtures thereof.

16. The device as in claim 13, wherein the polyol is selected from the group consisting of polycaprolactone polyols, polycarbonate polyols, polyester polyols, polytetrahydrofuran polyol, and mixtures thereof.

17. The device as in claim 13, wherein a catalyst is added to one of the two parts of the polymerizable material forming a polyurethane.

18. The device as in claim 17, wherein the catalyst is selected from the group consisting of tin esters, tin alkylesters, tin mercaptides, amines, tertiary amines, dibutyl tin dilaurate, and mixtures thereof.

19. The device as in claim 13, wherein low molecular weight diols are added to one part of the two-part polymerizable material forming a polyurethane.

20. The device as in claim 11, wherein the two-part polymerizable material has as one part mixtures of poly(hydroxyalkyl(meth)acrylates) and poly(alkyl(meth)acrylates) and as the other part polyfunctional (meth)acrylate monomers or oligomers.

21. The device as in claim 20, wherein the polymerizable material is cured using a free radical initiator and an amine activator.

22. The device as in claim 11, wherein the two-part polymerizable material has as one-part mixtures of tetra and trifunctional epoxy resin and as the other part a multifunctional amine or amino terminated elastomer.

23. The device as in claim 11, wherein the two-part polymerizable material is a polymer complex of polyanions or polycations.

24. The device as in claim 23, wherein the polymer complex of polyanions is selected from the group consisting of sodium carboxymethyl cellulose, sodium cellulose sulphate, sodium alginate, sodium hyaluronate, and mixtures thereof.

25. The device as in claim 23, wherein the polymer complex of polycations is selected from the group consisting of chitosan, quaternised chitosan, amino alkylated and subsequently quarternised cellulose, poly-L-lysine, and mixtures thereof.

26. The device as in claim 1, wherein the polymerizable material is a light activated polymerizable material.

27. The device as in claim 26, wherein the light activated polymerizable material comprises a mixture of a polyfunctional urethane acrylate or polyfunctional urethane methacrylate and a polyfunctional acrylate resin.

28. The device as in claim 26, wherein the light activated polymerizable material comprises a mixture of 2,2-bis-(4-(2-hydroxy-3-methacryloyloxypropoxy)phenyl) propane and a photoinitiation system.

29. The device as in claim 26, wherein the light activated polymerizable material comprises a one-part system of triisocyanates or higher isocyanates and OH-functional acrylates or methacrylates.

30. The device as in claim 1, wherein the polymerizable material is a heat activated polymerizable material.

31. The device as in claim 1, wherein the polymerizable material is in a state selected from the group consisting of a liquid, gel, colloid, paste, suspension, powder, grain, or granule.

32. The device as in claim 1, wherein the porous matrix is in the form of a woven or non-woven mesh, sheeting, braided or unbraided tubing, woven or non-woven fabric, or a sponge.

33. The device of claim 1, wherein the porous matrix is selected from the group consisting of polyethylenes including ultra high molecular weight polyethylenes, polyesters, polyetheretherketone, polyurethanes, polyesterurethane, polyester/polyol block copolymers, poly ethylene terepthalate, polytetrafluoro ethylene polyesters, nylons, polysulphanes, cellulose materials, polyaramids, carbon or glass fibers, polyvinyl chlorides, stryrenic resins, polypropylenes, polycarbonates, acrylonitrile-butadiene-styrene (“ABS”), acrylics, styrene acrylonitriles, and mixtures, copolymers, and mixtures thereof.

34. A method for intervertebral disc repair comprising:
inserting a porous matrix into an at least partially evacuated disc space;
injecting a polymerizable material into the porous matrix;
and allowing the polymerizable material to polymerize in situ.

35. A method as in claim 34, wherein the polymerizable material is injected into the porous matrix using a hypodermic needle or cannula.

36. A method as in claim 34, wherein allowing the polymerizable material to cure in situ comprises allowing body fluids to contact the polymerizable material, applying light to the polymerizable material, or applying heat to the polymerizable material.

37. A method for intervertebral disc repair comprising:
contacting a porous matrix with a polymerizable material;
inserting the porous matrix into an at least partially evacuated disc space; and
allowing the polymerizable material to polymerize in situ.

38. A method as in claim 37, wherein allowing the polymerizable material to cure in situ comprises allowing body fluids to contact the polymerizable material, applying light to the polymerizable material, or applying heat to the polymerizable material.

39. A method for intervertebral disc repair comprising:
contacting a porous matrix with saline solution or water;
contacting the porous matrix with a water activated polymerizable material;
inserting the porous matrix into an at least partially evacuated disc space; and
allowing the water activated polymerizable material to polymerize in situ.

40. A method for intervertebral disc repair comprising:
contacting a porous matrix with a first part of a two-part polymerizable material;
inserting the porous matrix into an at least partially evacuated disc space;
injecting the complementary second part of the two-part polymerizable material into the porous matrix; and
allowing the two-part polymerizable material to cure in situ.

41. The method as in claim 40, wherein the second part of the two-part polymerizable material is injected into the porous matrix using a hypodermic needle or cannula.

42. The method as in claim 34, wherein the disk space is evacuated by curettage, suction, laser nucleotomy, or chemonucleolysis.

43. The method as in claim 34, wherein the porous matrix is inserted into the evacuated disk space using a relatively small cannula and a flexible, semi-rigid push rod to push the matrix through the cannula.

44. A surgical kit, comprising:
a porous matrix,
a trimming device for sizing the porous matrix,
a polymerizable material, and
a device for injecting the polymerizable material.
